(19) United States
(12) Patent Application Publication
(10) Pub. No.: US 2026/0108474 A1
(43) Pub. Date: Apr. 23, 2026

(54) PAMAM-BASED NANOPARTICLE PROTEIN DEGRADATION SYSTEM AND METHOD OF PREPARATION AND USE THEREOF

(71) Applicant: QILU UNIVERSITY OF TECHNOLOGY (SHANDONG ACADEMY OF SCIENCES), Jinan (CN)

(72) Inventors: Yingshu GUO, Jinan (CN); Wennan YAN, Jinan (CN)

(21) Appl. No.: 19/426,262

(22) Filed: Dec. 19, 2025

(30) Foreign Application Priority Data
Aug. 7, 2025 (CN) 2025111013588

(51) Int. Cl. A61K 9/51; A61K 47/54; A61K 47/59; A61K 47/69; A61P 35/00

(52) U.S. Cl. CPC A61K 9/5146; A61K 9/5115; A61K 9/5192; A61K 47/542; A61K 47/545; A61K 47/59; A61K 47/6923; A61P 35/00

(57) ABSTRACT

A PAMAM-based protein degradation system and a method of preparation and use thereof are provided. The protein degradation system comprises: a silica nanoparticle core; and a poly(amidoamine) dendrimer (PAMAM) layer coated on a surface of the silica nanoparticle, wherein the PAMAM layer is linked via amide bonds to three small-molecule ligands: MDM2 protein ligand Idasanutlin, GLUT1 protein ligand Lavendustin B and E3 ubiquitin ligase ligand Thalidomide-NH—CH₂—COOH. The nanoparticle protein degradation system cooperatively degrades MDM2 protein and GLUT1 protein. This cooperative degradation strategy not only effectively suppresses proliferation and energy metabolism of tumor cells, but also significantly enhances the stability of p53 protein. By restoring the normal function of p53 protein, tumor cell growth is further inhibited, providing a new strategy for cancer therapy.

### CROSS-REFERENCE TO RELATED APPLICATIONS

The present application claims the priority to Chinese Patent Application No. CN202511101358.8, filed on Aug. 7, 2025, with the title “Protein Degradation System Based on Poly(amidoamine) Dendrimer and Method of Preparation and Use Thereof”. The entire disclosure of the above-identified application is incorporated herein by reference for all purposes.

### TECHNICAL FIELD

The present invention relates to the field of biomedicine technology, and in particular to a protein degradation system based on poly(amidoamine) dendrimer (PAMAM), and a method of preparation and use thereof.

### BACKGROUND OF THE INVENTION

The information disclosed in the background section is provided only to enhance understanding of the general background of the invention and is not necessarily to be construed as an admission that such information constitutes prior art known to a person skilled in the art.

The occurrence and development of malignant tumors involve aberrant expression and functional dysregulation of multiple key regulatory proteins, leading to uncontrolled cell proliferation and metabolic reprogramming. Among numerous regulatory factors, functional inactivation of certain key tumor suppressor proteins is a major driving factor in tumor progression. For example, an important tumor suppressor protein, such as p53, often loses its function due to the overexpression of its negative regulatory factor. The negative regulatory factor significantly reduces the intracellular level or activity of p53 protein by promoting its ubiquitination and degradation, thereby releasing the inhibition of tumor cell proliferation. Restoring the normal function of such key tumor suppressor proteins is one of the important strategies for cancer therapy.

However, for tumors that are complex diseases driven synergistically by multiple signaling pathways, protein degradation strategies targeting a single target are often limited in efficacy and are insufficient to fully suppress tumor growth, adaptation and drug resistance. In practice, multi-target cooperative degradation strategies also face considerable challenges.

### SUMMARY OF THE INVENTION

In view of the limitations of single-target protein degradation strategies in suppressing tumor cell proliferation and energy metabolism, there remains a need for a technical solution capable of coordinately regulating the expression of multiple key proteins at the nanoscale. The present invention provides a nanoparticle protein degradation system based on poly(amidoamine) dendrimer (PAMAM), and a method of preparation and use thereof. The nanoparticle protein degradation system can cooperatively degrade mouse double minute 2 (MDM2) protein and glucose transporter 1 (GLUT1) protein, not only loading ligands but also achieving the co-delivery of degradation ligands for MDM2 and GLUT1 proteins. This cooperative degradation strategy not only effectively inhibits tumor cell proliferation and energy metabolism, but also significantly enhances the stability of p53 protein. By restoring the normal function of p53 protein, the growth of tumor cells can be further suppressed, providing a new strategy for cancer therapy.

To achieve the above purpose, the present invention provides the following technical solutions.

In a first aspect, the present invention provides a protein degradation system based on a PAMAM, wherein the protein degradation system is in the form of a nanoparticle structure comprising silica (SiO₂) nanoparticles and a PAMAM layer coated on surfaces of the silica nanoparticles, and MDM2 protein ligands, GLUT1 protein ligands and E3 ubiquitin ligase ligands are linked via chemical bonds to the PAMAM layer.

MDM2 protein is a key regulatory protein in tumor cells, and its overexpression leads to abnormal proliferation of tumor cells. At the same time, overexpression of MDM2 protein results in down-regulation of p53 protein expression, thereby releasing the inhibitory effect of p53 protein on tumor cell proliferation. GLUT1 protein is a key transporter for glucose uptake in tumor cells, and its overexpression is closely associated with abnormal energy metabolism of tumor cells. In the protein degradation system of the present invention, MDM2 protein ligands and GLUT1 protein ligands are linked at the same time, such that the protein degradation system can specifically bind to MDM2 protein and GLUT1 protein. The E3 ubiquitin ligase ligands linked in the protein degradation system can specifically bind to an E3 ubiquitin ligase. In this case, through binding of the respective ligands, the protein degradation system can bring the E3 ubiquitin ligase into proximity to MDM2 protein and GLUT1 protein, thereby promoting precise recognition and interaction of the E3 ubiquitin ligase with MDM2 protein and GLUT1 protein, and consequently attaching ubiquitin molecules to the target proteins to gradually form polyubiquitin chains. Proteasomes can recognize the formed polyubiquitin chains and degrade MDM2 protein and GLUT1 protein. By degrading MDM2 protein, the normal function of p53 protein can be restored, tumor cell proliferation can be suppressed, and the sensitivity of tumor cells to the system can be enhanced. By degrading GLUT1 protein, the energy metabolism of tumor cells can be inhibited and their survival capability can be weakened. In addition, the E3 ubiquitin ligase ligands can further enhance the specificity of the entire degradation system by precisely directing the E3 ubiquitin ligase to the target proteins, thereby minimizing impacts on non-target proteins, effectively reducing potential side effects and significantly improving drug safety.

In order to enable the MDM2 protein ligands, GLUT1 protein ligands and E3 ubiquitin ligase ligands to exert their functions, the present invention selects silica nanoparticles having a surface coated with a PAMAM layer as a carrier. In this carrier, the highly branched dendritic structure and abundant surface active groups (such as amino groups) of PAMAM, in combination with the high specific surface area, stable chemical properties and easily modifiable characteristics of SiO₂, significantly increase loading density and ligand-binding capability, so that ligands can be efficiently loaded and multi-target degradation can be achieved.

In another aspect, the present invention provides a method for preparing a protein degradation system based on a PAMAM, comprising the steps of:
providing silica nanoparticles and carrying out carboxylation modification on surfaces of the silica nanoparticles;
subjecting the silica nanoparticles having surfaces subjected to carboxylation modification to an amidation reaction with a PAMAM so that the PAMAM coats the silica nanoparticles; and
subjecting the silica nanoparticles having surfaces coated with the PAMAM to an amidation reaction with MDM2 protein ligands, GLUT1 protein ligands and E3 ubiquitin ligase ligands so that the MDM2 protein ligands, GLUT1 protein ligands and E3 ubiquitin ligase ligands are linked to the PAMAM on the surfaces of the silica nanoparticles.

In a third aspect, the present invention provides use of the above protein degradation system based on a PAMAM in the preparation of an anti-tumor drug.

In a fourth aspect, the present invention provides a method for treating a tumor, comprising administering, to a subject in need thereof, a drug comprising the above protein degradation system based on a PAMAM.

The beneficial effects of the present invention are as follows:
according to the present invention, silica nanoparticles having a surface coated with a PAMAM layer are used to efficiently load MDM2 protein ligands, GLUT1 protein ligands and E3 ubiquitin ligase ligands, so that an E3 ubiquitin ligase can be brought into proximity to MDM2 protein and GLUT1 protein, thereby promoting precise recognition and interaction of the E3 ubiquitin ligase with MDM2 protein and GLUT1 protein and consequently attaching ubiquitin molecules to the target proteins to gradually form polyubiquitin chains. Through this proteasome pathway, MDM2 protein is degraded to restart the normal function of p53 protein, and GLUT1 protein is degraded to inhibit the energy metabolism of tumor cells, thereby achieving a cooperative anti-tumor effect. Through such cooperative degradation, the nanoparticle system can more effectively inhibit proliferation of tumor cells and provides a new strategy for tumor therapy.

### BRIEF DESCRIPTION OF THE DRAWINGS

The accompanying drawings, which constitute a part of the present specification, serve to provide a further understanding of the present invention. The exemplary embodiments of the invention and descriptions thereof are intended to illustrate the invention and are not to be construed as unduly limiting the invention.

FIG. 1A is a transmission electron microscopy (TEM) image of SiO₂ nanoparticles prepared in Example 1 of the present invention, with a scale bar of 100 nm.

FIG. 1B is a TEM image of SG3TAC nanoparticles prepared in Example 1 of the present invention, with a scale bar of 100 nm.

FIG. 2A is a dynamic light scattering (DLS) data graph of SG3TAC nanoparticles (B) prepared in Example 1 of the present invention.

FIG. 2B is another DLS data graph of SG3TAC nanoparticles (B) prepared in Example 1 of the present invention.

FIG. 3 shows Zeta potential results of SG3 nanoparticles prepared in Example 1 of the present invention.

FIG. 4 shows Fourier-transform infrared (FT-IR) spectra of SG3 nanoparticles prepared in Example 1 of the present invention.

FIG. 5 shows FT-IR spectra of SG3TAC nanoparticles prepared in Example 1 of the present invention.

FIG. 6 shows results of cytotoxicity of different materials prepared in Example 1 and Comparative Examples 1-2 of the present invention on lung cancer cells.

FIG. 7 shows fluorescence images of live and dead A549 cells incubated with different materials prepared in Example 1 and Comparative Examples 1-2 of the present invention. Calcein-AM (calcein acetoxymethyl ester) and propidium iodide (PI) were used as fluorescent dyes. “Merge” denotes merged images. The scale bar represents 100 μm.

FIG. 8 shows western blot (WB) results of MDM2 protein in A549 cells treated with SG3-CM prepared in Comparative Example 1 of the present invention at different concentrations.

FIG. 9 shows WB results of MDM2 protein in A549 cells treated with SG3-CM prepared in Comparative Example 1 of the present invention for different times.

FIG. 10 shows WB results of MDM2 protein in A549 cells treated with different inhibitors in the present invention.

FIG. 11 shows WB results of MDM2 protein in A549 cells treated with different materials in the present invention.

FIG. 12 shows immunofluorescence analysis results of MDM2 protein expression in A549 cells treated with different materials in the present invention. The scale bar represents 20 μm.

FIG. 13 shows WB results of GLUT1 protein in A549 cells treated with SG3-CG prepared in Comparative Example 2 of the present invention at different concentrations.

FIG. 14 shows WB results of GLUT1 protein in A549 cells treated with SG3-CG prepared in Comparative Example 2 of the present invention for different times.

FIG. 15 shows results of ATP levels in A549 cells treated with different drugs in the present invention.

FIG. 16 shows WB results of GLUT1 protein in A549 cells treated with different inhibitors in the present invention.

FIG. 17 shows WB results of GLUT1 protein in A549 cells treated with different materials in the present invention.

FIG. 18 shows immunofluorescence analysis results of GLUT1 protein expression in A549 cells treated with different materials in the present invention. The scale bar represents 20 μm.

FIG. 19 shows results of MDM2 protein and GLUT1 protein in A549 cells treated with different concentrations of SG3TAC for 12 hours in the present invention.

FIG. 20 shows WB results of p53 protein in A549 cells treated with different materials in the present invention.

FIG. 21 shows immunofluorescence analysis results of p53 protein expression in A549 cells treated with different materials in the present invention. The scale bar represents 20 μm.

FIG. 22 shows WB results of apoptosis-related proteins BAX, PUMA and NOXA in A549 cells under different treatments in the present invention.

### DETAILED DESCRIPTION OF THE INVENTION

It should be understood that the following detailed description is exemplary and is provided for further illustration of the present invention. Unless otherwise indicated, all technical and scientific terms used herein have the same meaning as commonly understood by a person of ordinary skill in the art to which the present invention pertains.

It should be noted that the terminology used herein is for the purpose of describing particular embodiments only and is not intended to be limiting of the exemplary embodiments of the invention. As used herein, unless the context clearly indicates otherwise, the singular forms also include the plural forms. Furthermore, it should be understood that when the terms “comprise” and/or “include” are used in this specification, they specify the presence of features, steps, operations, devices, components and/or combinations thereof.

In view of the limited anti-cancer efficacy of single-target drug degradation and the difficulty in achieving the desired anti-cancer effect with multi-target cooperative drug degradation, the present invention provides a protein degradation system based on a PAMAM, and a method of preparation and use thereof.

In a typical embodiment of the present invention, a PAMAM-based protein degradation system is provided. The protein degradation system is in the form of a nanoparticle structure. The nanoparticle structure comprises silica nanoparticles and a PAMAM layer coated on surfaces of the silica nanoparticles, and MDM2 protein ligands, GLUT1 protein ligands and E3 ubiquitin ligase ligands are linked via chemical bonds to the PAMAM layer.

In some embodiments, a PAMAM-based protein degradation system is provided, wherein the protein degradation system is in the form of a nanoparticle structure, and the nanoparticle structure comprises:
a core; and
a coating layer;
wherein the core is a silica nanoparticle, the coating layer is a PAMAM layer coated on surfaces of the core, and the PAMAM layer is linked via amide bonds to the following three small-molecule ligands: an MDM2 protein ligand Idasanutlin, a GLUT1 protein ligand Lavendustin B and an E3 ubiquitin ligase ligand Thalidomide-NH—CH₂—COOH.

In some embodiments, the nanoparticle structure has a particle size of 220-270 nm.

In some embodiments, the silica nanoparticles have a particle size of 200-250 nm.

In some embodiments, the PAMAM is a generation 3 PAMAM with amino surface groups (PAMAM-G3-NH₂).

In some embodiments, the silica nanoparticles and the PAMAM layer coated on surfaces of the silica nanoparticles together form PAMAM-coated silica nanoparticles, and a mass ratio of the silica nanoparticles to the PAMAM used to form the PAMAM layer is 100:20.0-21.0.

In some embodiments, a mass ratio of the PAMAM-coated silica nanoparticles, the E3 ubiquitin ligase ligand, the MDM2 protein ligand and the GLUT1 protein ligand is 10:2.3-2.4:1-2:1-2.

In another embodiment of the present invention, a method for preparing a PAMAM-based protein degradation system is provided, comprising:
providing silica nanoparticles and carrying out carboxylation modification on surfaces of the silica nanoparticles;
subjecting the silica nanoparticles having surfaces subjected to carboxylation modification to an amidation reaction with PAMAM so that the PAMAM coats the silica nanoparticles; and
subjecting the PAMAM-coated silica nanoparticles to an amidation reaction with the MDM2 protein ligands, the GLUT1 protein ligands and the E3 ubiquitin ligase ligands so that the MDM2 protein ligands, the GLUT1 protein ligands and the E3 ubiquitin ligase ligands are linked to the PAMAM on the surfaces of the silica nanoparticles.

In some embodiments, the method comprises:
providing silica nanoparticles and carrying out carboxylation modification on surfaces of the silica nanoparticles to obtain carboxylated silica nanoparticles;
subjecting the carboxylated silica nanoparticles to an amidation reaction with PAMAM in the presence of a condensing agent to form a PAMAM layer on surfaces of the silica nanoparticles, thereby obtaining PAMAM-coated silica nanoparticles; and
subjecting the PAMAM-coated silica nanoparticles to respective amidation reactions with ligands Thalidomide-NH—CH—COOH, Idasanutlin and Lavendustin B in the presence of the condensing agent such that the three ligands are linked via amide bonds to the PAMAM layer.

In some embodiments, the carboxylation modification on the surfaces of the silica nanoparticles comprises: first performing amino-modification on the surfaces of the silica nanoparticles, and then converting amino groups into carboxyl groups by reaction with a diacid anhydride. Specifically, 3-aminopropyltrimethoxysilane is used to perform amino-modification on the surfaces of the silica nanoparticles. Specifically, the amino-modification is carried out at a temperature of 110-130° C. Specifically, the amino-modification is carried out for 22-26 h. The diacid anhydride referred to in the present invention is a cyclic anhydride formed by intramolecular dehydration of an organic dicarboxylic acid containing two carboxyl groups, such as succinic anhydride and glutaric anhydride. The diacid anhydride can react with amino groups to form amide bonds and generate free carboxyl groups, thereby converting the amino groups into carboxyl groups. Specifically, a mass ratio of the diacid anhydride to the amino-modified silica nanoparticles is 14-16:1.

In the present invention, the purpose of subjecting the silica nanoparticles having surfaces subjected to carboxylation modification to an amidation reaction with PAMAM is to coat PAMAM on the surfaces of the silica nanoparticles via formation of amide bonds. In order to improve the efficiency and stability of PAMAM coating, in some embodiments, N-(3-dimethylaminopropyl)-N′-ethylcarbodiimide hydrochloride (EDC) and N-hydroxysuccinimide (NHS) are used to activate carboxyl groups on the surfaces of the silica nanoparticles, followed by addition of PAMAM to carry out the amidation reaction.

In some embodiments, a mass ratio of the silica nanoparticles to PAMAM is 100:20.0-21.0. Under this ratio, PAMAM can better cover the surfaces of the silica nanoparticles and is more favorable for subsequent efficient loading of various ligands.

In the present invention, the purpose of subjecting the PAMAM-coated silica nanoparticles to an amidation reaction with the MDM2 protein ligands, the GLUT1 protein ligands and the E3 ubiquitin ligase ligands is to link the ligands to the PAMAM on the surfaces of the silica nanoparticles via formation of amide bonds. In order to better link the various ligands, in some embodiments, EDC and NHS are used to activate carboxyl groups in the MDM2 protein ligands, the GLUT1 protein ligands and the E3 ubiquitin ligase ligands, followed by addition of the PAMAM-coated silica nanoparticles to carry out the amidation reaction. Specifically, the amidation reaction is carried out at a temperature of 20-30° C. for 1-3 days.

In some embodiments, a mass ratio of the PAMAM-coated silica nanoparticles, the E3 ubiquitin ligase ligands, the MDM2 protein ligands and the GLUT1 protein ligands is 10:2.3-2.4:1-2:1-2.

In a third embodiment of the present invention, use of the above PAMAM-based protein degradation system in the preparation of an anti-tumor drug is provided.

In one embodiment, an anti-tumor drug is provided, which comprises the PAMAM-based protein degradation system.

In some embodiments, the tumor is lung cancer. Specifically, the tumor is non-small cell lung cancer. Specifically, the tumor cells are A549 cells.

In a fourth embodiment of the present invention, a method for treating a tumor is provided, comprising administering to a subject in need thereof a therapeutically effective amount of the above PAMAM-based protein degradation system or an anti-tumor drug comprising the PAMAM-based protein degradation system.

The administration route employed in the present invention for administering to the subject may be intravenous, intraperitoneal, intramuscular, subcutaneous, spinal or another parenteral administration route. Parenteral administration refers to a route of administration that is typically carried out by injection rather than enteral or topical administration, including but not limited to intravenous, intraperitoneal, intramuscular, intra-arterial, intrasheath, intralymphatic, intralesional, intracavitary, intraorbital, intracardiac, intradermal, intratracheal, subcutaneous, subepidermal, intra-articular, subcapsular, subarachnoid, intraspinal, epidural and intra-sternal injection and infusion. Alternatively, non-parenteral routes may be used, such as local, cutaneous or mucosal administration, for example intranasal, oral, vaginal, rectal, sublingual or topical administration.

In some embodiments, the subject is a human or an animal. Specifically, the animal may be a mouse, rat, dog, rabbit, pig or the like.

In some embodiments, the tumor is lung cancer. Specifically, the tumor is non-small cell lung cancer. Specifically, the tumor cells are A549 cells.

To enable those skilled in the art to more clearly understand the technical solutions of the present invention, the invention will be described in detail below with reference to specific embodiments and comparative examples.

In the following embodiments, the materials and reagents used are as follows.

3-Aminopropyltrimethoxysilane (APTS), anhydrous toluene, succinic anhydride (SA, 99%), N-(3-dimethylaminopropyl)-N′-ethylcarbodiimide hydrochloride (EDC) and N-hydroxysuccinimide (NHS) were purchased from Sigma-Aldrich (St. Louis, MO, USA). Acetone, anhydrous ethanol, aqueous ammonia, dimethyl sulfoxide (DMSO, 99%), methanol and skim milk powder were purchased from Shanghai Macklin Biochemical Co., Ltd. (Shanghai, China). Trypsin-EDTA (0.25%) was purchased from Gibco (Thermo Fisher Scientific, Waltham, MA, USA). 20×TBST buffer, fetal bovine serum (FBS), DMEM, a penicillin-streptomycin antibiotic mixture and GAPDH antibody were obtained from Beijing Solarbio Science & Technology Co., Ltd. (Beijing, China). 4′,6-Diamidino-2-phenylindole (DAPI), phenylmethylsulfonyl fluoride (PMSF), 3-(4,5-dimethyl-2-thiazolyl)-2,5-diphenyl-2H-tetrazolium bromide (MTT), calcein acetoxymethyl ester (Calcein-AM), propidium iodide (PI), SDS lysis buffer, bovine serum albumin (BSA), BCA protein assay kit, immunostaining permeabilization solution (Triton X-100), horseradish peroxidase (HRP)-conjugated goat anti-rabbit IgG (H+L) and HRP-conjugated goat anti-mouse IgG (H+L) were purchased from Beyotime Biotechnology (Shanghai, China). PAGE gel rapid preparation kit, two-color pre-stained protein marker and polyvinylidene difluoride (PVDF) membranes (0.45 μm) were purchased from Epizyme Biotech (Shanghai Epizyme Biomedical Technology Co., Ltd., Shanghai, China). PVDF membranes (0.22 μm) were purchased from Millipore (Billerica, MA, USA). Generation 3 poly(amidoamine) dendrimer with amino surface groups (PAMAM-G3-NH₂) was purchased from Weihai Chenyuan Molecular New Materials Co., Ltd. (Weihai, China). The E3 ubiquitin ligase ligand Thalidomide-NH—CH₂—COOH (CAS No. 927670-97-1), the MDM2 protein ligand Idasanutlin (CAS No. 1229705-06-9), the GLUT1 ligand Lavendustin B (CAS No. 125697-91-8), MG132 and MLN4924 were purchased from MedChemExpress (MCE). The MDM2 antibody was purchased from Wuhan Huamei Bioengineering Co., Ltd. (Wuhan, China). The GLUT1 antibody was purchased from Hangzhou Huaan Biotechnology Co., Ltd. (Hangzhou, China). The p53 antibody was purchased from Wuhan Sanying Biotechnology Co., Ltd. (Wuhan, China). BAX and PUMA antibodies were purchased from Abcam (Cambridge, UK). The NOXA antibody was purchased from Abways (Shanghai, China). Ultra-sensitive ECL chemiluminescence kit was purchased from NCM Biotech (Suzhou, China). Alexa Fluor 555-conjugated goat anti-rabbit IgG and Alexa Fluor 488-conjugated goat anti-mouse IgG were purchased from Sangon Biotech (Shanghai) Co., Ltd. (Shanghai, China). A549 lung cancer cells were obtained from Wuhan Pricella Biotechnology Co., Ltd. (Wuhan, China). ATP assay kit was obtained from Nanjing Jiancheng Bioengineering Institute (Nanjing, China).

The instruments used in the following examples include: zeta potential and nanoparticle size analyzer (Zetasizer Nano, Malvern Instrument Ltd., UK); transmission electron microscope (TEM) (JEM2100, Thermo Fisher, Japan); Fourier-transform infrared (FT-IR) spectrometer (TENSOR, Bruker, Germany); chemiluminescence imaging system (ChemiScope 6200, CLiNX, China); fluorescence microscope (Ti2-E, Nikon, Japan); a microplate reader (K3 TOUCH, Thermo Fisher, China); electrophoresis cell (Mini-PROTEAN® system, Bio-Rad, USA); and protein transfer apparatus (Mini Trans-Blot cell, Bio-Rad, USA).

### Example 1

### Preparation of a PAMAM-Based Protein Degradation System

1. Preparation of PAMAM-Coated silica Nanoparticles

### (1) Preparation of Monodisperse Spherical Silica (SiO₂) Nanoparticles by Stöber-Fink-Bohn Method.

A mixture of 500 μL aqueous ammonia (25 w/w %), 14 mL ethanol and 2 mL deionized water was stirred at 50° C. for 20 min. Then 500 μL tetraethyl orthosilicate (TEOS) was dropwise added into the above solution and the mixture was continuously stirred at 50° C. for 1.5 h. After the reaction, the product was collected by centrifugation (4000-14000 rpm, 10 min), washed three times with ethanol and deionized water, and dried at 70° C. for 12 h to obtain silica nanoparticles.

### (2) Coating of PAMAM on Silica Nanoparticles.

0.2 g of SiO₂ was dispersed in 20 mL anhydrous toluene containing 0.4 mL APTS and stirred at 120° C. for 24 h. After centrifugation, the obtained solid was washed successively with ultrapure water and methanol and dried under vacuum at 50° C. to obtain SiO₂—NH₂. Subsequently, 0.2 g of SiO₂—NH₂ was dispersed in 20 mL acetone, followed by addition of 3 g succinic anhydride and reaction for 24 h. After the reaction, the product was dried under vacuum at 50° C. to obtain a carboxylated product SiO₂—COOH. Then, the obtained SiO₂—COOH was dispersed in 5 mL DMSO. EDC (46 mg, dissolved in 5 mL DMSO) and NHS (27.6 mg, dissolved in 5 mL DMSO) were sequentially added to activate the carboxyl groups. Under stirring, a solution of G3 PAMAM dendrimer (PAMAM-G3-NH₂ dendrimer, 41.6 mg in 5 mL DMSO) was added and the reaction was continued for 1 day. The reaction mixture was then centrifuged at 12000 rpm for 10 min three times, and the precipitate was lyophilized to obtain PAMAM-coated SiO₂ nanoparticles (SG3).

### 2. Preparation of the PAMAM-Based Protein Degradation System

NHS (45.0 mg in 5 mL DMSO), EDC (74.0 mg in 5 mL DMSO), E3 ubiquitin ligase ligand (11.7 mg in 5 mL DMSO), MDM2 protein ligand (10 mg in 5 mL DMSO) and GLUT1 protein ligand (10 mg in 5 mL DMSO) were mixed and incubated at 25° C. for 2 h. SG3 particles (50.0 mg in 5 mL DMSO) were slowly added dropwise into the above mixture and the resulting mixture was stirred at 25° C. for 2 days. After the reaction, the product was collected by centrifugation at 12000 rpm for 10 min, washed three times with a “water-ethanol” mixed solvent by centrifugation, and lyophilized to obtain SG3TAC.

### Example 2

Example 2 was the same as Example 1 except that:

### 2. Preparation of the PAMAM-Based Protein Degradation System

NHS (45.0 mg in 5 mL DMSO), EDC (74.0 mg in 5 mL DMSO), E3 ubiquitin ligase ligand (11.7 mg in 5 mL DMSO), MDM2 protein ligand (10 mg in 5 mL DMSO) and GLUT1 protein ligand (10 mg in 5 mL DMSO) were mixed and incubated at 25° C. for 3 h. SG3 particles (50.0 mg in 5 mL DMSO) were slowly added dropwise into the above mixture and the mixture was stirred at 25° C. for 3 days. The product was then collected by centrifugation at 12000 rpm for 10 min, washed three times with a “water-ethanol” mixed solvent by centrifugation, and lyophilized to obtain the final product.

### Example 3

Example 3 was the same as Example 1 except that:

### 2. Preparation of the PAMAM-Based Protein Degradation System

NHS (45.0 mg in 5 mL DMSO), EDC (74.0 mg in 5 mL DMSO), E3 ubiquitin ligase ligand (11.7 mg in 5 mL DMSO), MDM2 protein ligand (5 mg in 5 mL DMSO) and GLUT1 protein ligand (5 mg in 5 mL DMSO) were mixed and incubated at 25° C. for 1 h. SG3 particles (50.0 mg in 5 mL DMSO) were slowly added dropwise into the mixture and the reaction was carried out at 25° C. for 1 day. The product was collected by centrifugation at 12000 rpm for 10 min, washed three times with a “water-ethanol” mixed solvent by centrifugation, and lyophilized to obtain the final product.

### Comparative Example 1

Comparative Example 1 was the same as Example 1 except that:

### 2. Preparation of the PAMAM-Based Protein Degradation System

NHS (45.0 mg in 5 mL DMSO), EDC (74.0 mg in 5 mL DMSO), E3 ubiquitin ligase ligand (11.7 mg in 5 mL DMSO) and MDM2 protein ligand (10 mg in 5 mL DMSO) were mixed and incubated at 25° C. for 2 h. SG3 particles (50.0 mg in 5 mL DMSO) were then added and slowly dropped into the above mixture, followed by stirring at 25° C. for 2 days. The product was collected by centrifugation at 12000 rpm for 10 min, washed three times with a “water-ethanol” mixed solvent by centrifugation, and lyophilized to obtain SG3-CM.

### Comparative Example 2

Comparative Example 2 was the same as Example 1 except that:

### 2. Preparation of the PAMAM-Based Protein Degradation System

NHS (45.0 mg in 5 mL DMSO), EDC (74.0 mg in 5 mL DMSO), E3 ubiquitin ligase ligand (11.7 mg in 5 mL DMSO) and GLUT1 protein ligand (10 mg in 5 mL DMSO) were mixed and incubated at 25° C. for 2 h. SG3 particles (50.0 mg in 5 mL DMSO) were then added and slowly dropped into the above mixture, followed by stirring at 25° C. for 2 days. The product was collected by centrifugation at 12000 rpm for 10 min, washed three times with a “water-ethanol” mixed solvent by centrifugation, and lyophilized to obtain SG3-CG.

### Comparative Example 3

Comparative Example 3 was the same as Example 1 except that:

### 2. Preparation of the PAMAM-Based Protein Degradation System

NHS (45.0 mg in 5 mL DMSO), EDC (74.0 mg in 5 mL DMSO) and E3 ubiquitin ligase ligand (11.7 mg in 5 mL DMSO) were mixed and incubated at 25° C. for 2 h. SG3 particles (50.0 mg in 5 mL DMSO) were then added and slowly dropped into the above mixture, followed by stirring at 25° C. for 2 days. The product was collected by centrifugation at 12000 rpm for 10 min, washed three times with a “water-ethanol” mixed solvent by centrifugation, and lyophilized to obtain SG3-C.

### Comparative Example 4

Comparative Example 4 was the same as Example 1 except that:

### 2. Preparation of the PAMAM-Based Protein Degradation System

NHS (45.0 mg in 5 mL DMSO), EDC (74.0 mg in 5 mL DMSO) and MDM2 protein ligand (10 mg in 5 mL DMSO) were mixed and incubated at 25° C. for 2 h. SG3 particles (50.0 mg in 5 mL DMSO) were then slowly added dropwise into the above mixture, followed by stirring at 25° C. for 2 days. The product was collected by centrifugation at 12000 rpm for 10 min, washed three times with a “water-ethanol” mixed solvent by centrifugation, and lyophilized to obtain SG3-M.

### Comparative Example 5

Comparative Example 5 was the same as Example 1 except that:

### 2. Preparation of the PAMAM-Based Protein Degradation System

NHS (45.0 mg in 5 mL DMSO), EDC (74.0 mg in 5 mL DMSO) and GLUT1 protein ligand (10 mg in 5 mL DMSO) were mixed and incubated at 25° C. for 2 h. SG3 particles (50.0 mg in 5 mL DMSO) were then slowly added dropwise into the above mixture, followed by stirring at 25° C. for 2 days. The product was collected by centrifugation at 12000 rpm for 10 min, washed three times with a “water-ethanol” mixed solvent by centrifugation, and lyophilized to obtain SG3-G.

### Test Example 1

### Cytotoxicity of Different Materials on Lung Cancer Cells

A549 lung cancer cells were seeded into 96-well plates and incubated at 37° C. and 5% CO₂ for 12 h to allow cell attachment. Then, 20 μL of SG3, 20 μL of SG3-CM, 20 μL of SG3-CG or 20 μL of SG3TAC at different concentrations were added to each well, and incubation was continued for 12 h. After that, 100 μL of MTT solution (5 mg/mL) was added to each well and incubation was continued for 4 h. The supernatant in the 96-well plate was removed and 100 μL of DMSO was added to each well. The absorbance at 492 nm was then recorded.

### Test Example 2

### Study on Degradation Conditions

A549 cells were cultured in 6-well plates at a density of 2×10⁶ cells per well for 24 h.

To investigate the relationship between the concentration of SG3-CM, SG3-CG or SG3TAC and the protein degradation effect, SG3-CM, SG3-CG or SG3TAC was diluted with DMEM to a concentration range of 0-50 μg/mL, and 40 μL of the resulting solution was co-incubated with cells for 12 h.

To study the effect of incubation time of SG3-CM, SG3-CG or SG3TAC with cells on protein degradation effect, 40 μL of SG3-CM solution at 40 μg/mL, 40 μL of SG3-CG solution at 40 μg/mL, or 40 μL of SG3TAC solution at 40 μg/mL was co-incubated with cells for 0-24 h.

To investigate the degradation pathway of SG3-CM or SG3-CG, cells were treated with MG132 or MLN4924 for 1 h, and then 40 μL of SG3-CM solution at 40 μg/mL or 40 μL of SG3-CG solution at 40 μg/mL was added to different wells and incubation was continued at 37° C. for 48 h.

Cells obtained in the above three experiments were washed twice with ice-cold PBS and lysed with 150-200 μL of SDS lysis buffer (containing 1 mM protease inhibitor cocktail). Protein quantification was performed using a BCA protein assay kit. The protein samples were mixed with 5×SDS-PAGE loading buffer, boiled for 30 min, and then 20-40 μg of protein per lane was loaded on 10% or 12% SDS-PAGE gels. Electrophoresis was carried out using a protein electrophoresis apparatus for 1-2 h at 80 V. A protein transfer apparatus was used to transfer the proteins from the gels onto PVDF membranes (0.45 μm or 0.22 μm) at 120 mA for 1 h. The membranes were blocked with 25 mL of 5% skim milk in TBST at room temperature for 1 h, followed by washing three times with TBST. The membranes were then incubated with MDM2 antibody (1:2000), GLUT1 antibody (1:50000), GAPDH antibody (1:50000), p53 antibody (1:25000), BAX antibody (1:5000), PUMA antibody (1:3000) or NOXA antibody (1:2000) at 4° C. for 12 h. After washing three times (10 min each) with TBST buffer, the membranes were incubated with HRP-conjugated goat anti-rabbit IgG (H+L) (1:1000) and HRP-conjugated goat anti-mouse IgG (1:1000) at room temperature for 1 h. Finally, the membranes were washed three times (10 min each) with TBST buffer, and chemiluminescence development was performed using an ECL substrate. Western blot bands were detected using a chemiluminescence imaging system.

### Test Example 3

### Feasibility Study on Protein Degradation by SG3-CM and SG3-CG

A549 cells were cultured in confocal culture dishes and incubated at 37° C. and 5% CO₂ for 12 h to allow cell attachment. PBS, SG3, SG3-CM, SG3-CG or SG3TAC was added to different dishes, and incubation was continued for 12 h. Cells were then washed three times with PBS buffer and fixed with anhydrous methanol for 15 min. After that, cells were incubated with 0.5% Triton X-100 at room temperature for 10 min. After washing with PBS buffer, 3% BSA solution was added and the cells were incubated at room temperature for 2 h. After washing with PBS buffer, MDM2 antibody, GLUT1 antibody or p53 antibody was added, followed by incubation at 4° C. overnight. After washing with PBS buffer, Alexa Fluor 488-conjugated goat anti-mouse IgG or Alexa Fluor 555-conjugated goat anti-rabbit IgG was added and incubation was carried out at room temperature for 1 h. After washing with PBS buffer, the cells were stained with DAPI for 15 min. After another PBS buffer wash, images were acquired using a fluorescence microscope.

### Results and Discussion

### 1. Characterization of SiO₂, SG3, SG3-CM, SG3-CG and SG3TAC Nanoparticles

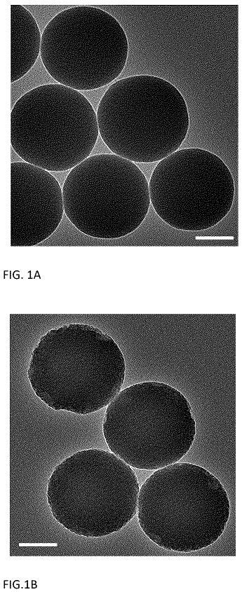

The morphology of SiO₂ and SG3TAC nanoparticles was characterized by TEM, as shown in FIG. 1. The prepared SiO₂ and SG3TAC exhibited uniform morphology and good dispersity. Compared with SiO₂ nanoparticles shown in FIG. 1A, SG3TAC nanoparticles exhibited rougher surfaces as shown in FIG. 1B, which is attributed to the coverage by PAMAM that decreased the surface smoothness.

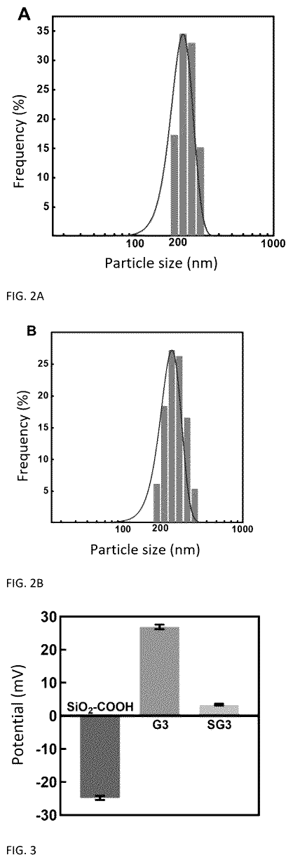

The particle size of SiO₂ nanoparticles was about 230 nm, which is consistent with the DLS data shown in FIG. 2A. The particle size of SG3TAC nanoparticles was about 250 nm, also consistent with the DLS data shown in FIG. 2B.

Surface potential analysis, as shown in FIG. 3, revealed that the carboxylated SiO₂ nanoparticles carried a negative surface potential (−24.8±0.62 mV). After loading PAMAM, the particle surface exhibited a positive potential (3.34±0.27 mV), due to the abundant amino groups on PAMAM which reversed the surface potential.

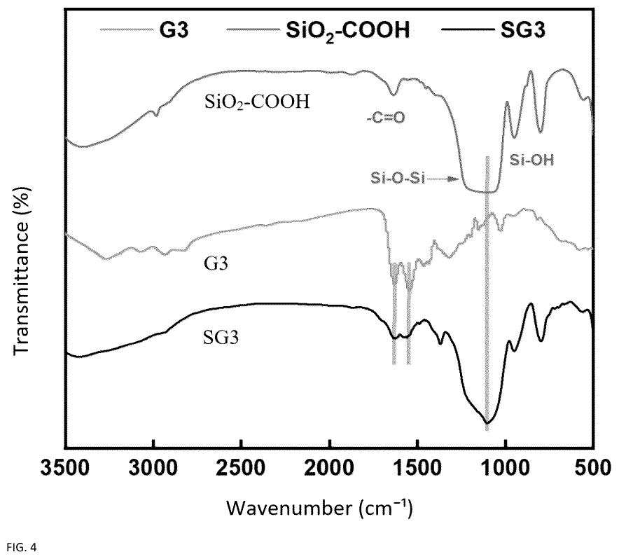

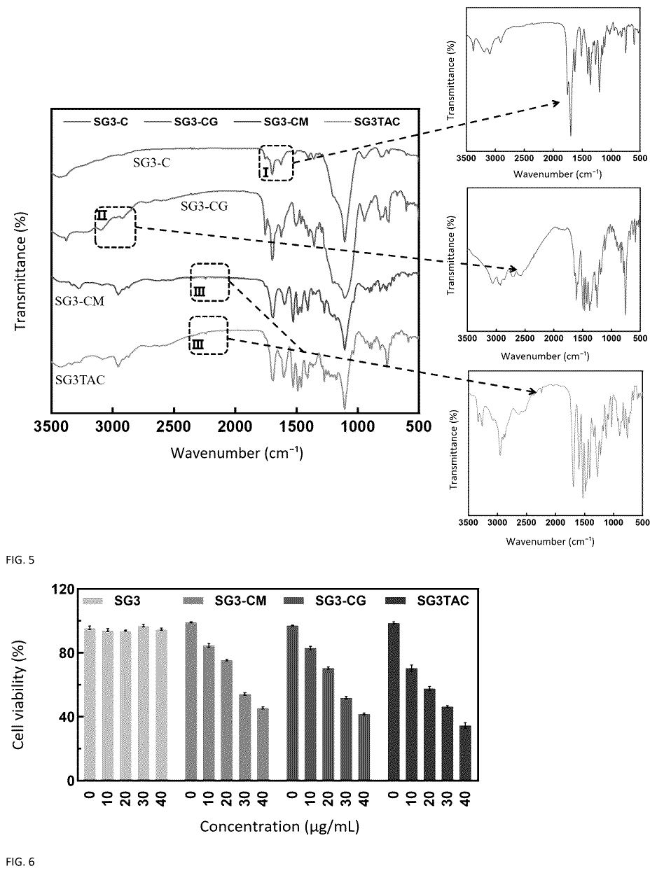

FT-IR analysis was used to characterize the functional groups on the nanoparticle surface, as shown in FIG. 4. In the IR spectrum of carboxylated SiO₂ nanoparticles, distinct absorption peaks appeared at 960 cm⁻¹, 1020-1110 cm⁻¹ and 1640 cm⁻¹, which are attributed to the asymmetric bending and stretching vibration of Si—OH, the asymmetric stretching vibration of Si—O—Si, and the vibration of —COOH, respectively. In the IR spectrum of PAMAM, an absorption peak appeared at 1560-1640 cm⁻¹, corresponding to the N—H bending vibration. In the IR spectrum of SG3, prominent absorption peaks appeared at 960 cm⁻¹, 1020-1110 cm⁻¹ and 1560-1640 cm⁻¹, confirming that SG3 is silica nanoparticles with amino-rich surfaces.

The IR spectrum of the product (SG3-C) obtained after reaction of SG3 with the E3 ubiquitin ligase ligand is shown in FIG. 5, in which absorption peaks appeared at 1700-2000 cm-corresponding to the characteristic triplet peaks of a benzene ring. After reaction of SG3-C with the GLUT1 ligand, the product SG3-CG exhibited an absorption peak at 3200-3500 cm⁻¹, corresponding to O—H stretching vibration in the GLUT1 ligand. After reaction of SG3-C with the MDM2 ligand, the product SG3-CM exhibited an absorption peak at 2210-2260 cm⁻¹, corresponding to the characteristic C N triple bond peak of the MDM2 ligand. After reaction of SG3-C with both the GLUT1 ligand and the MDM2 ligand, the resulting SG3TAC exhibited absorption peaks at 595 cm⁻¹ and 3200-3500 cm⁻¹ similar to those of SG3-CG, and an absorption peak at 2210-2260 cm⁻¹ similar to that of SG3-CM.

### 2. Evaluation of Cytotoxicity of SG3, SG3-CM, SG3-CG and SG3TAC Nanoparticles

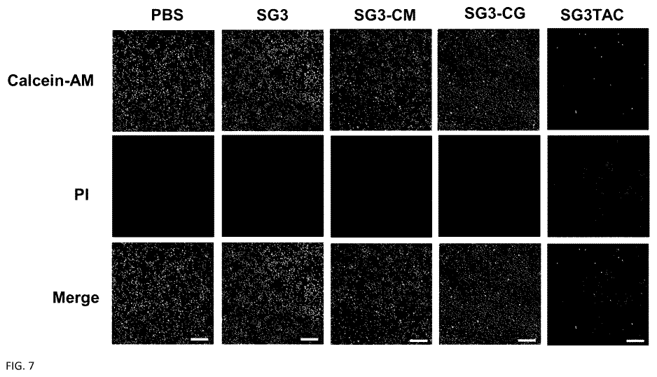

The viability of A549 cells treated with SG3, SG3-CG, SG3-CM and SG3TAC was assessed using the MTT assay. As shown in FIG. 6, SG3 exhibited no obvious effect on the viability of A549 cells in the concentration range of 0-40 μg/mL, indicating good biocompatibility. For SG3-CG, SG3-CM and SG3TAC, the viability of A549 cells decreased gradually with increasing concentration (0-40 μg/mL). Among them, SG3TAC showed the most pronounced inhibitory effect on A549 cells, indicating stronger anti-tumor activity.

Further live/dead cell assays of A549 cells showed that, relative to the PBS group, the number of live cells (green fluorescence) markedly decreased and the number of dead cells (red fluorescence) markedly increased in the SG3TAC-treated group. The proportion of dead cells in the SG3TAC-treated group was significantly higher than that in the SG3-CM and SG3-CG groups, as shown in FIG. 7. These results are consistent with the MTT assay and together confirm the cytotoxicity and anti-proliferative effect of SG3TAC.

### 3. Evaluation of MDM2 Protein Degradation by SG3-CM

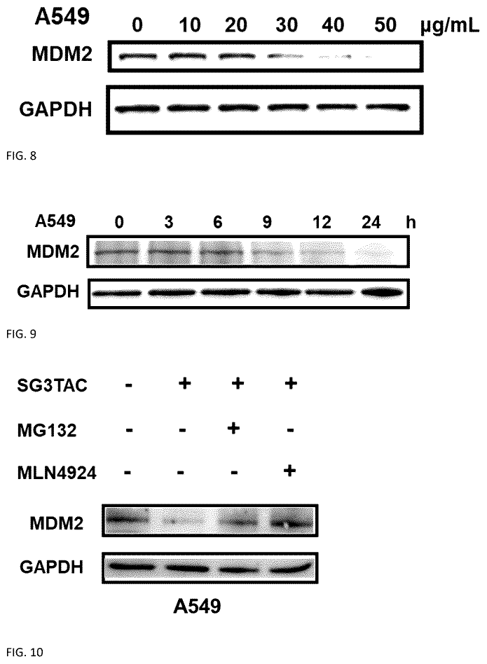

The degradation of MDM2 protein in A549 cells was analyzed by western blot. A549 cells were exposed to SG3-CM nanoparticles at different concentrations (0-50 μg/mL) for 12 h. As shown in FIG. 8, the expression level of MDM2 protein gradually decreased with increasing concentration of SG3-CM, displaying a concentration-dependent degradation behavior. In particular, at higher concentrations (40 μg/mL and 50 μg/mL), the band intensity of MDM2 protein was significantly lower than that of the control group (0 μg/mL), indicating that SG3-CM effectively promoted the degradation of MDM2 protein at these concentrations.

To evaluate the effect of exposure time on MDM2 protein expression, A549 cells were exposed to 40 μg/mL SG3-CM for 0-24 h. As shown in FIG. 9, the band intensity of MDM2 protein gradually decreased after 9 h, demonstrating the strong degradation capability of SG3-CM.

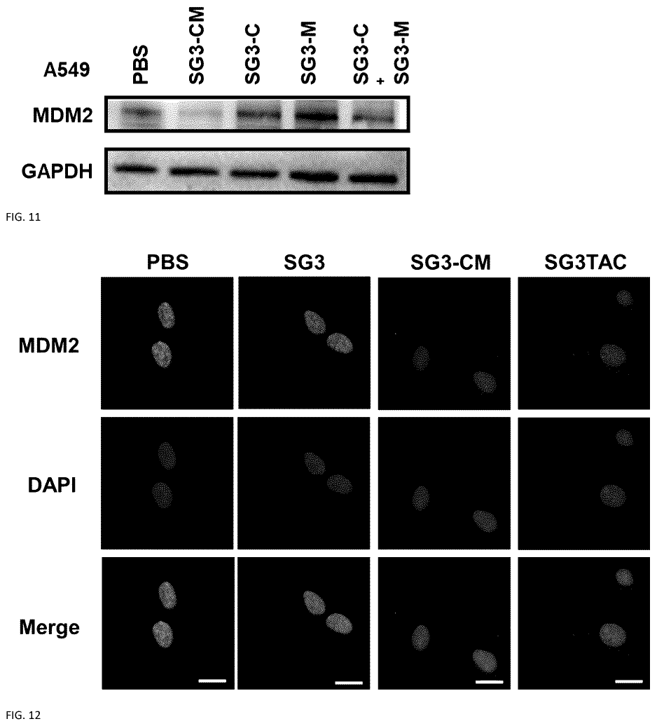

On this basis, to further explore the degradation pathway of SG3-CM, the proteasome inhibitor MG132 and the NEDD8-activating enzyme (NAE) inhibitor MLN4924 were used. As shown in FIG. 10, compared with A549 cells directly exposed to SG3-CM, pre-treatment of A549 cells with MG132 followed by exposure to SG3-CM significantly inhibited the degradation of MDM2 protein, indicating that SG3-CM-mediated MDM2 protein degradation is related to the proteasome pathway. Pre-treatment of A549 cells with MLN4924 followed by exposure to SG3-CM also significantly inhibited MDM2 protein degradation, indicating that SG3-CM-mediated MDM2 protein degradation is also related to the ubiquitination pathway.

Meanwhile, the effects of SG3-C, SG3-M (without E3 ubiquitin ligase ligand) and a mixture thereof on MDM2 protein degradation in A549 cells were investigated. As shown in FIG. 11, in contrast to SG3-CM, almost no MDM2 protein degradation occurred in the SG3-C group, SG3-M group and mixture group. This result fully demonstrates that only SG3-CM can effectively induce MDM2 protein degradation. It also indirectly indicates that a ternary complex formed by PAMAM, MDM2 protein and E3 ubiquitin ligase in A549 cells underlies the mechanism for MDM2 protein degradation.

In addition, immunofluorescence imaging of MDM2 protein in cells was performed using Alexa Fluor 488-conjugated goat anti-mouse IgG. As shown in FIG. 12, A549 cells treated with SG3-CM or SG3TAC exhibited a marked reduction in green fluorescence signal, indicating the high efficiency of SG3-CM and SG3TAC in regulating MDM2 protein expression.

### 4. Evaluation of GLUT1 Protein Degradation by SG3-CG

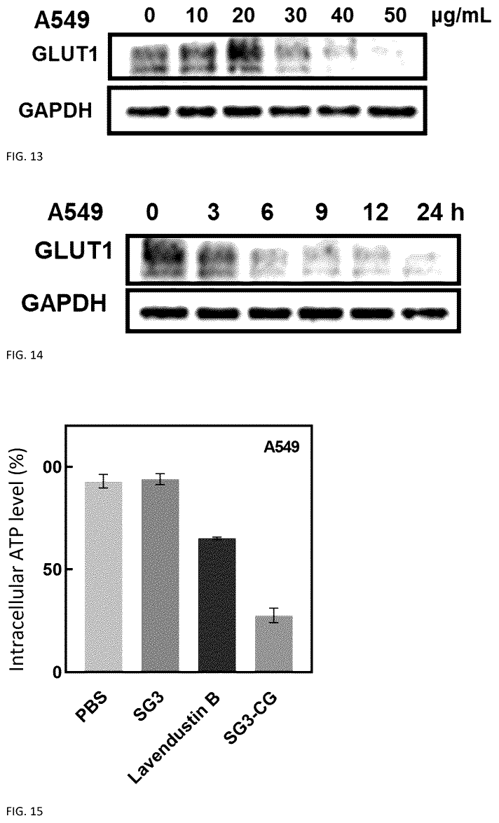

The degradation of GLUT1 protein in A549 cells was analyzed by western blot. A549 cells were exposed to SG3-CG nanoparticles at different concentrations (0-50 μg/mL) for 12 h. As shown in FIG. 13, the expression level of GLUT1 protein gradually decreased with increasing concentration of SG3-CG, displaying a concentration-dependent degradation trend. In particular, at higher concentrations (40 μg/mL and 50 μg/mL), the band intensity of GLUT1 protein was significantly lower than that of the control group (0 μg/mL), indicating that SG3-CG effectively promoted the degradation of GLUT1 protein at these concentrations.

To evaluate the effect of exposure time on GLUT1 protein expression, A549 cells were exposed to 40 μg/mL SG3-CG for 0-24 h. As shown in FIG. 14, the band intensity of GLUT1 protein gradually decreased after 6 h, demonstrating the strong degradation capability of SG3-CG.

Meanwhile, intracellular ATP levels were measured and found to be significantly reduced in SG3-CG-treated cells, as shown in FIG. 15. This indicates that SG3-CG effectively inhibited the function of GLUT1 protein and reduced glucose uptake, thereby affecting ATP production and suggesting that it exerts anti-tumor effects by interfering with cellular energy metabolism.

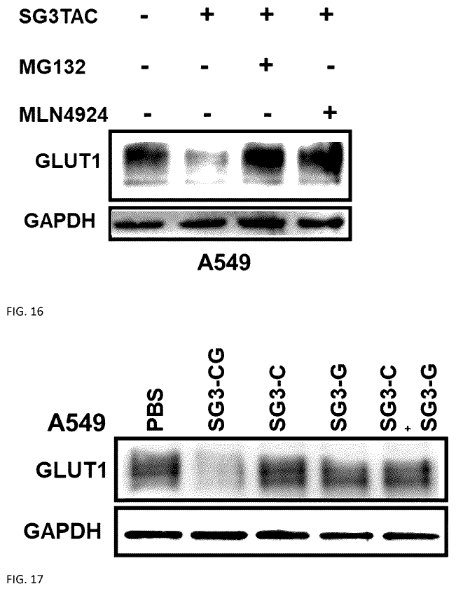

On this basis, to further explore the degradation pathway of SG3-CG, the proteasome inhibitor MG132 and the NAE inhibitor MLN4924 were used. As shown in FIG. 16, compared with A549 cells directly exposed to SG3-CG, pre-treatment with MG132 followed by exposure to SG3-CG significantly inhibited GLUT1 protein degradation, indicating that SG3-CG-mediated GLUT1 protein degradation is related to the proteasome pathway. Pre-treatment with MLN4924 followed by exposure to SG3-CG also significantly inhibited GLUT1 protein degradation, indicating that SG3-CG-mediated GLUT1 protein degradation is also related to the ubiquitination pathway.

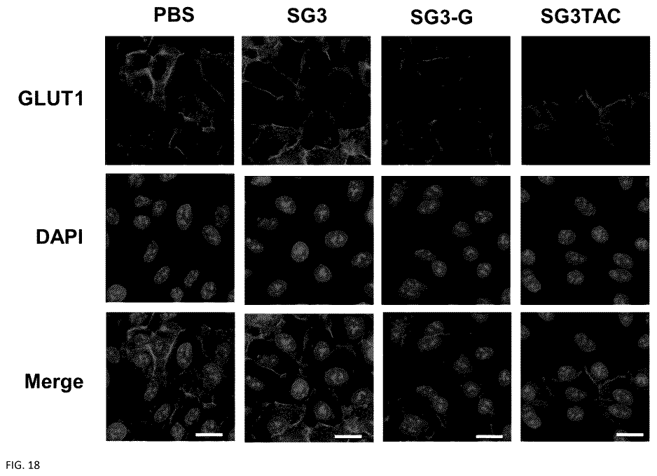

Meanwhile, the effects of SG3-C, SG3-G (without E3 ubiquitin ligase ligand) and a mixture thereof on GLUT1 protein degradation in A549 cells were investigated. As shown in FIG. 17, in contrast to SG3-CG, almost no GLUT1 protein degradation occurred in the SG3-C group, SG3-G group and mixture group. This result fully demonstrates that only SG3-CG can effectively induce GLUT1 protein degradation. It also indirectly indicates that a ternary complex formed by PAMAM, GLUT1 protein and E3 ubiquitin ligase in A549 cells underlies the mechanism for GLUT1 protein degradation.

In addition, immunofluorescence imaging of GLUT1 protein in cells was performed using Alexa Fluor 555-conjugated goat anti-rabbit IgG. As shown in FIG. 18, A549 cells treated with SG3-CG or SG3TAC exhibited markedly weakened red fluorescence signals, indicating the high efficiency of SG3-CG and SG3TAC in regulating GLUT1 protein expression.

### 5. Effect of SG3TAC Nanoparticles on p53 Protein Expression

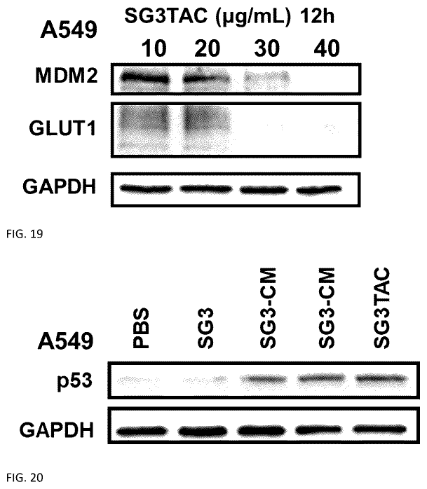

As shown in FIG. 19, A549 cells were treated with SG3TAC nanoparticles at different concentrations (10-40 μg/mL) for 12 h. Both GLUT1 protein and MDM2 protein exhibited significant concentration-dependent changes in expression levels. Specifically, the expression of GLUT1 protein gradually decreased with increasing SG3TAC concentration and was almost completely suppressed at 30 μg/mL, indicating a remarkable degradation effect of SG3TAC on GLUT1 protein. Similarly, the expression of MDM2 protein also showed a concentration-dependent decrease after SG3TAC treatment and reached the lowest level at the highest concentration (40.0 μg/mL).

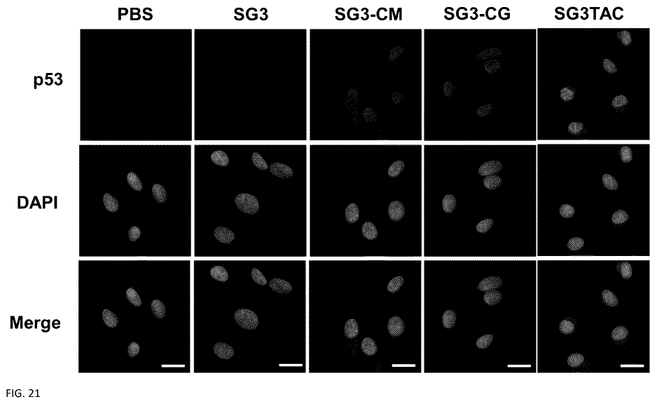

As shown in FIG. 20, p53 protein expression in A549 cells was investigated. Compared with the PBS group, SG3, SG3-CM, SG3-CG and SG3TAC treatments all altered the expression level of p53 protein in A549 cells. SG3 had little effect on p53 expression, whereas SG3-CM and SG3-CG treatments increased p53 expression. Notably, SG3TAC treatment led to the highest expression of p53 protein, indicating that SG3TAC can more effectively up-regulate p53 protein expression.

Moreover, immunofluorescence imaging of p53 protein in cells was performed using Alexa Fluor 555-conjugated goat anti-rabbit IgG. As shown in FIG. 21, SG3-CM, SG3-CG and SG3TAC treatments significantly enhanced the red fluorescence signal, indicating that these treatments effectively promoted p53 protein expression. Among them, SG3TAC treatment resulted in the strongest red fluorescence signal of p53 protein, demonstrating its high efficiency in inducing p53 protein expression.

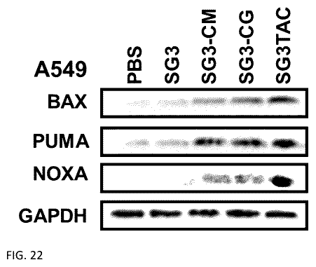

The expression levels of apoptosis-related proteins BAX, PUMA and NOXA in cells are shown in FIG. 22. Compared with the control group, treatment with SG3-CM, SG3-CG and SG3TAC up-regulated the expression levels of BAX, PUMA and NOXA in A549 cells. Specifically, both SG3-CM and SG3-CG treatments increased the expression of these proteins, while SG3TAC treatment further significantly increased the expression levels of BAX, PUMA and NOXA, reaching the highest levels. This indicates that SG3TAC has a stronger ability to induce expression of these pro-apoptotic proteins. These results suggest that SG3TAC can more effectively activate the p53 signaling pathway, up-regulate pro-apoptotic proteins and thereby enhance the apoptosis process.

In summary, SG3TAC nanoparticles significantly reduced the expression levels of MDM2 protein and GLUT1 protein in a concentration-dependent manner and effectively enhanced p53 protein expression, demonstrating their potential in modulating key protein expression in tumor cells.

The foregoing description is merely preferred embodiments of the present invention and is not intended to limit the invention. For a person skilled in the art, various modifications and changes can be made to the present invention. Any modification, equivalent replacement or improvement made within the spirit and principle of the invention shall be included within the scope of protection of the invention.

1. A protein degradation system based on a poly(amidoamine) dendrimer (PAMAM), the system being in the form of a nanoparticle structure, the nanoparticle structure comprising:
a core; and
a coating layer;
wherein the core is a silica nanoparticle, and the coating layer is a PAMAM layer coated on a surface of the core, and the PAMAM layer is linked via amide bonds to three small-molecule ligands: an MDM2 protein ligand Idasanutlin, a GLUT1 protein ligand Lavendustin B, and an E3 ubiquitin ligase ligand Thalidomide-NH—CH₂—COOH.

2. The protein degradation system of claim 1, wherein the nanoparticle structure has a particle size of 220-270 nm.

3. The protein degradation system of claim 1, wherein the silica nanoparticle core has a particle size of 200-250 nm.

4. The protein degradation system of claim 1, wherein the PAMAM is a generation 3 poly(amidoamine) dendrimer with amino surface groups (PAMAM-G3-NH₂).

5. The protein degradation system of claim 1, wherein the silica nanoparticles and a PAMAM layer coated on surfaces of the silica nanoparticles together form PAMAM-coated silica nanoparticles, and a mass ratio of the silica nanoparticles to PAMAM used to form the PAMAM layer is from 100:20.0 to 100:21.0.

6. The protein degradation system of claim 5, wherein a mass ratio of the PAMAM-coated silica nanoparticles, the E3 ubiquitin ligase ligand, the MDM2 protein ligand and the GLUT1 protein ligand is 10:(2.3-2.4):(1-2):(1-2).

7. A method for preparing the protein degradation system of claim 1, comprising:
providing silica nanoparticles and carrying out carboxylation modification on surfaces of the silica nanoparticles to obtain carboxylated silica nanoparticles;
subjecting the carboxylated silica nanoparticles to an amidation reaction with PAMAM in the presence of a condensing agent to form a PAMAM layer on surfaces of the silica nanoparticles, thereby obtaining PAMAM-coated silica nanoparticles; and
subjecting the PAMAM-coated silica nanoparticles to respective amidation reactions with ligands Thalidomide-NH—CH₂—COOH, Idasanutlin and Lavendustin B in the presence of the condensing agent, such that the three ligands are linked via amide bonds to the PAMAM layer.

8. The method of claim 7, wherein the carboxylation modification comprises: performing amino-modification on surfaces of the silica nanoparticles, and then converting amino groups into carboxyl groups by reaction with a diacid anhydride.

9. The method of claim 7, wherein the carboxylation modification comprises:
(a) performing amino-modification on surfaces of the silica nanoparticles using 3-aminopropyltrimethoxysilane to obtain amino-modified silica nanoparticles; and
(b) reacting the amino-modified silica nanoparticles with a diacid anhydride to convert surface amino groups into carboxyl groups.

10. The method of claim 8, wherein the diacid anhydride is succinic anhydride or glutaric anhydride.

11. The method of claim 7, wherein the condensing agent comprises N-(3-dimethylaminopropyl)-N′-ethylcarbodiimide hydrochloride and N-hydroxysuccinimide.

12. The method of claim 7, wherein the amidation reactions between the PAMAM-coated silica nanoparticles and ligands Thalidomide-NH—CH—COOH, Idasanutlin and Lavendustin B are carried out at 20-30° C. for 1-3 days.

13. An anti-tumor drug comprising a protein degradation system of claim 1.

14. The anti-tumor drug of claim 13, wherein the tumor is lung cancer.

15. The anti-tumor drug of claim 13, wherein the tumor is non-small cell lung cancer.

16. A method of treating a tumor, comprising administering to a subject in need thereof a therapeutically effective amount of a protein degradation system of claim 1 or an anti-tumor drug comprising the protein degradation system.

17. The method of claim 16, wherein the tumor is lung cancer.

18. The method of claim 16, wherein the tumor is non-small cell lung cancer.
